(19) United States
(12) Patent Application Publication
(10) Pub. No.: US 2015/0153061 A1
(43) Pub. Date: Jun. 4, 2015

(54) METHOD FOR CONTROLLING AND MONITORING THE LEVEL OF CONFINEMENT OF INTERNAL AIR, AND RELATED ENVIRONMENT DEVICE AND STATION

(71) Applicant: CENTRE SCIENTIFIQUE TECHNIQUE DU BATIMENT, Champs Sur Marne (FR)

(72) Inventors: Jacques Henri Riberon, Le Perreux Sur Marne (FR); Olivier Ramaho, Mareuilles-Les-Meaux (FR)

(21) Appl. No.: 14/412,314

(22) PCT Filed: Jun. 19, 2013
(86) PCT No.: PCT/FR2013/051426

§ 371 Date: Dec. 31, 2014

(30) Foreign Application Priority Data
Jul. 2, 2012 (FR) 1256327

(51) Int. Cl. F24F 11/00; G01N 33/00

(52) U.S. Cl. CPC F24F 11/0012; G01N 33/004; F24F 11/0001; F24F 11/0015; F24F 2011/0041

(57) ABSTRACT

The invention relates to a method for controlling and monitoring the level of confinement of the internal environment of a room, which includes determining the confinement index I from measurements of the CO₂ content during a predetermined period, and reducing the level of confinement in accordance with the CO₂ content. According to the invention, periods of occupancy of the internal environment are determined by means of at least one presence sensor provided with a calibration device which enables the sensor to react to the presence of humans as from an occupancy threshold. The invention also relates to a device configured in particular to calculate the confinement index I.

### TECHNICAL FIELD OF THE INVENTION

The invention relates to the field of methods and devices for regulating and controlling an internal environment.

“Internal environment” means a closed space such as a room where gaseous exchanges with the outside are limited.

The quality of air present inside a room depends both on the quality of the external air and the characteristics of the rooms in question. The internal sources of contaminants are due to human presence (human bioeffluent such as microbiological agents or volatile organic compounds) and human activity (for example tobacco smoke or volatile organic compounds). The internal sources may also come from combustion appliances giving off in particular contaminants such as carbon monoxide, nitrogen oxides and aldehydes; or construction or furnishing materials giving off contaminants such as formaldehyde, volatile organic compounds or fibres. Moreover, the hygrometric conditions of buildings may promote the development of microbiological contaminants such as acarid allergens or moulds. These substances, breathed in by persons, may cause a simple nuisance, for example due to the odour, or an irritation. There may also cause pathologies such as asthma, in particular in populations at risk, such as children, elderly persons or sick persons, whose respiratory systems are under development or weakened. They may cause acute intoxications or more serious illnesses such as cancer.

In this field, devices comprising sensors such as carbon dioxide (CO₂) sensors are normally used by a person specialising in the exposure assessment of interior environments.

### PRIOR ART

Thus, through the document FR 2 945 335, a method and device for regulating and controlling the confinement of internal air of buildings are known.

However, this method and device are based on the calculation of a confinement index during declared periods of occupation.

This system is suited to schools and day nurseries, which are establishments where presence may be fairly well recorded. However, declarations of presence are sometimes difficult to collect from the occupants of buildings and may in some cases not be sufficiently precise to provide an exact calculation of the confinement index.

The use of a presence sensor that triggers CO₂ measurements is mentioned. However, this system is not entirely satisfactory since detections may take place when the environment is occupied fortuitously by a single person, which falsifies the calculations since the confinement must be representative of the exposure of children. This is because the presence detector does not make it possible to know the number of persons present in a room.

### DISCLOSURE OF THE INVENTION

The invention aims to remedy the drawbacks of the prior art and in particular to propose a method for regulating and controling the level of confinement of an internal environment, comprising:
determination of a confinement index I, comprising
i) measurements of the CO₂ level at regular time intervals during determined occupation periods;
ii) at the end of the measurements of the CO₂ level, the determination of a confinement index I from said measurements;
iii) repetition of steps i) and ii);
as soon as a measurement of the CO₂ level exceeds a predetermined threshold S₂. reduction in the level of confinement until a CO₂ level is reached equal to a predetermined threshold S₁, less than S₂, this reduction in the level of confinement being effected during the period of measurement of the CO₂ level as many times as a CO₂ measurement exceeds the threshold S₂.

According to a first aspect, the periods of occupation of the internal environment are determined by means of at least one presence sensor equipped with a calibration device enabling it to react to human presence as from an occupation threshold. Thus the method according to the invention allows an evaluation of the index when the detection exceeds a predetermined threshold.

“Period of occupation” means a period of presence of at least one physical person inside the room in question. The occupation threshold preferably corresponds to at least half the theoretical number of persons expected inside the room in question. To do this, the presence detector is equipped with a calibration device enabling it to react to the human presence in a relevant manner. Thus detections below the threshold will not be considered in the measurement of CO₂ and/or the calculation of the confinement index.

For example, the periods of occupation are determined by a single presence sensor during a predetermined time threshold.

Advantageously, several presence sensors communicate with each other and/or with a computing means for determining the periods of occupation.

In a variant, the occupation can be estimated using sensors disposed at different points in the room in question, in order to carry out a more reliable detection.

According to another variant, the information from the sensors is hierarchised according to a law of the master/slave type. Naturally other types of hierarchisation can be envisaged without departing from the scope of the invention.

Preferably, said at least one presence sensor is a passive infrared sensor.

Preferably, the method comprises a transmission of data relating to the index I, to a remote management centre.

The invention also relates to a device for regulating and mcontrolling the level of confinement of internal air used according to the method as described previously; the device comprising a CO₂ sensor, data recording means, computing means, preferably signalling means, characterised in that it is connected to at least one presence sensor equipped with a calibration device enabling it to react to human presence as from an occupation threshold.

According to a variant, the device comprises at least one temperature, humidity and/or pressure sensor.

Another subject matter of the invention consists of a portable environment station comprising a device for controlling and monitoring the level of confinement of the internal environment of a room, which itself comprises a CO₂ sensor and data recording means, configured to compute a confinement index I, characterised in that it comprises means for displaying retrospective information relating to the confinement of the interior environment.

Preferably, the portable environment station described above comprises signalling means, preferably LEDs, for instantaneous management of the ventilation conditions of the internal environment.

Thus this device is preferentially designed for regulating and controlling the confinement of the air inside accommodation. It allows instantaneous management of the ventilation conditions of an accommodation by means of luminous LEDs. Advantageously, retrospectively, the invention makes it possible to know the levels of confinement of the air during the night by displaying a history of the data.

### BRIEF DESCRIPTION OF THE FIGURES

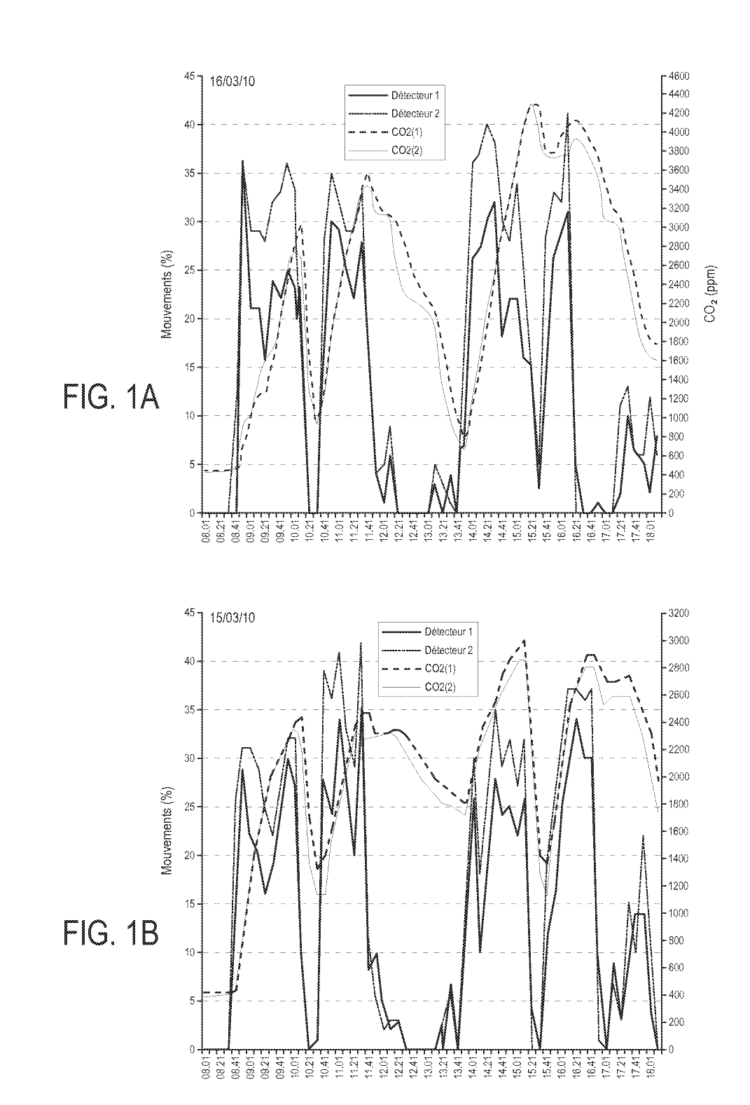

Other features, details and advantages of the invention will emerge from a reading of the following description, with reference to the appended FIGS. 1A and 1B, which illustrate the change in movement percentages of two presence sensors disposed in parallel in a classroom. FIG. 1A concerns a first day from 8.00 am to 6.00 pm while FIG. 1B concerns a second day from 8.00 am to 6.00 pm.

### DETAILED DESCRIPTION OF AN EMBODIMENT

The application FR 2 945 335 describes numerous aspects related to the method and device for controlling and regulating an internal environment other than those detailed below.

The main aim of the invention is to be able to easily associate a movement detector with the other means of the device according to the invention, for improved calculation of the confinement index I.

The device according to the invention is based on a confinement module, the main functionalities of which are as follows:

Firstly, the confinement module is configured so as to measure CO₂ levels in the air using the CO₂ sensor, preferably every minute.

In parallel with the measurement of CO₂, occupation is measured by at least one external presence sensor. The presence sensor is provided and connected to the confinement module. A cable or wireless connection can be provided for this purpose.

Secondly, the confinement module registers an average of measurements of CO₂ levels and measurements from the presence sensor, preferably the last ten measurements.

Thirdly, the confinement module computes and stores confinement indices I during parameterisable operating ranges, in particular according to the presence detected.

In order to characterise the periods of occupation, the presence sensor coupled to the device according to the invention may be a passive infrared movement sensor. For example, an HAA52N movement sensor sold under the brand name Velleman® may be used.

Preferably a so-called “CO₂ measurement, calculation and recording” mode is executed automatically at regular intervals, for example every minute, whatever the mode in which the confinement module is situated. The actions executed in this mode are as follows:

For each recording the following data are stored in the memory:

1. Time and date to the format YY/MM/DD/HH/MM/SS;

2. Average concentration of CO₂ over the last ten samples (in parts per million ppm);

3. Confinement index I, referred to as “ICONE”, if the module is in an operating range, otherwise the default aberrant value XX. For example an aberrant used is 255;

4. Number of seconds during which a presence was detected over the last ten minutes (in number or percentage according to the configuration of the sensor);

5. Average value supplied by the external presence sensor or sensors (optionally).

Thus, outside the occupation ranges, an aberrant value XX is allocated to the confinement index in order to take into account only the period of occupation.

Occupation is preferably estimated on the basis of a detection threshold during a given period.

Thus, in the presence of the movement sensor (Presence_sensor_active=1), the operating range is defined when the value of the sensor is greater than the presence threshold previously configured (Presence_sensor_threshold).

The value “Presence_sensor_active” makes it possible to define whether the presence sensor is being used and the index is calculated (1: sensor used, and 0: sensor not used).

The value “Presence_sensor_threshold” refers to the threshold, for example in tens of seconds, above which the module considers that a presence is detected. Every ten measurements (10 minutes for example), the confinement module will consider that a presence is detected if the sensor has detected a presence during more than “Presence_sensor_threshold”×10 seconds. Another reference period can be envisaged without departing from the scope of the invention. If the movement sensor is configured so as to express a result as a percentage, then the “Presence_sensor_threshold) is also expressed as a percentage.

In this way, the last value of the confinement index I referred to as “ICONE” displayed outside the aberrant values XX (for example 255), corresponds to the actual confinement index over the whole of the recording period.

By way of illustration, the threshold value (Presence_sensor_threshold) for defining a typical occupation of a classroom has been studied and defined at 10%. Below this threshold, it can be concluded that there is no occupation of the classroom by a “normal” complement, for example children in case of a classroom.

In the use of the device described in the application FR 2 945 335, the non-allocation of aberrant values to the confinement index I and the non-resetting to zero of the computation causes an estimation of the confinement index that takes into account all the prior values and because of this falsifies the calculations.

Differently and advantageously, the invention also proposes a module for resetting the confinement index I such that the confinement indices are calculated taking account only of the CO₂ values for the current day.

The appended figures (FIGS. 1A and 1B) illustrate examples of changes in the movement values as a percentage [Mvt(%)] of two presence sensors disposed in parallel in a classroom, over two days, from 8.00 am to 6.00 pm. The change in the amounts of movements recorded by the first movement detector D1 is illustrated in a solid bold line, while the change in the second D2 is in a dot and dash line. The change in the CO₂ levels in ppm [CO₂ (ppm)] recorded by the first CO₂ sensor (1) is in a dotted line while the change in the second CO₂ sensor (2) is in a fine line.

As can be seen in these figures, even if the detection curves follow substantially parallel changes, they cannot be superimposed, which shows firstly the importance of the determination of the detection thresholds for each detector. Secondly, these curves advantageously show that a better estimation of the occupation of the internal environment can be made using information from at least two movement sensors.

Thus it is possible to use, in a variant, a plurality of presence sensors communicating with one another for estimating the occupation of the internal environment, and thus avoid unwanted detections. The detection threshold and detection period values can be chosen so as to be different for the various detectors.

The invention also relates to a weather station comprising a program for calculating a confinement index I, and a display of the history of the indices. This type of weather station is particularly suited to habitat.

The user can for example have information on the confinement index 1 of his accommodation as well as information relating to at least one of the following parameters: temperature, pressure, humidity, weather forecasts, etc. Naturally all or some of this information is supplied by specific sensors and/or by a transmission of data, for example via the internet.

Numerous combinations can be envisaged without departing from the scope of the invention; a person skilled in the art would choose one or other according to the economic, ergonomic, dimensional or other constraints that he will have to comply with.

### KEY TO THE FIGURES

| | |
|---|---|
| Mouvements (%) | Movements (%) |

FIG. 1A

| | |
|---|---|
| Détecteur | Detector |

FIG. 1B

| | |
|---|---|
| Détecteur | Detector |

1. Method for regulating and controlling the level of confinement of an internal environment of a local, comprising:
determination of a confinement index I, comprising
i) measurements of the CO₂ level at regular time intervals during determined occupation periods;
ii) at the end of the measurements of the CO₂ level, the determination of a confinement index I from said measurements;
iii) repetition of steps i) and ii);
as soon as a measurement of the CO₂ level exceeds a predetermined threshold S₂, reduction in the level of confinement until a CO₂ level is reached equal to a predetermined threshold S₁, less than S₂, this reduction in the level of confinement being effected during the period of measurement of the CO₂ level as many times as a CO₂ measurement exceeds the threshold S₂,
characterised in that periods of occupation of the internal environment are determined by means of at least one presence sensor equipped with a calibration device enabling it to react to human presence as from an occupation threshold.

2. Method according to claim 1, characterised in that several presence sensors communicate with one another and/or with a computing means for determining periods of occupation.

3. Method according to claim 2, characterised in that the information from said at least one sensor is hierarchised according to a law of the master/slave type.

4. Method according to claim 1, characterised in that said at least one sensor is a passive infrared sensor.

5. Method according to claim 1, characterised in that it comprises a transmission of data related to the index I, to a remote management centre.

6. Device for regulating and controlling the level of confinement of the internal environment of a local used according to the method of claim 1, the device comprising a CO₂ sensor, data recording means, computing means, and preferably signalling means, characterised in that it is connected to at least one presence sensor equipped with a calibration device enabling it to react to human presence as from an occupation threshold.

7. Device according to claim 6, characterised in that it comprises at least one temperature, humidity and/or pressure sensor.
